(19) United States
(12) Patent Application Publication
(10) Pub. No.: US 2022/0315605 A1
(43) Pub. Date: Oct. 6, 2022

(54) IMPROVED PROCESS FOR THE PREPARATON OF PURE (1'R,6R,6aR,7R,13S,14S,16R)-5-(ACETYLOXY)-3',4',6,6a,7,13,14,16-OCTAHYDRO-6',8,14-TRIHYDROXY-7',9-DIMETHOXY-4,10,23-TRIMETHYLSPIRO[6,16-(EPITHIOPROPANOXYMETH ANO)-7,13-IMINO-12H-1,3-DIOXOLO[7,8]ISOQUINO[3,2-b][3]BENZAZOCINE-20,1'(2'H)-ISOQUINOLIN]-19-ONE POLYMORPH THERE OF

(71) Applicants: Thirumalai Rajan SRINIVASAN, Sangareddy (Dist), Hyderabad, Telangana (IN); MSN LABORATORIES PRIVATE LIMITED, R&D CENTER, Sangareddy (Dist), Hyderabad, Telangana (IN)

(72) Inventors: Thirumalai Rajan SRINIVASAN, Hyderabad, Telangana (IN); Eswaraiah SAJJA, Hyderabad, Telangana (IN); Rajeshwar Reddy SAGYAM, Hyderabad, Telangana (IN); Navin Kumar Reddy KESHAVAREDDY, Hyderabad, Telangana (IN)

(73) Assignee: MSN LABORATORIES PRIVATE LIMITED, R&D CENTER, Sangareddy (Dist), Hyderabad, Telangana (IN)

(21) Appl. No.: 17/623,041

(22) PCT Filed: Jun. 29, 2020
(86) PCT No.: PCT/IN2020/050564

§ 371 Date: Dec. 27, 2021

(30) Foreign Application Priority Data
Jun. 27, 2019 (IN) 201941025538
Sep. 21, 2019 (IN) 201941038170

(51) Int. Cl. C07D 515/22

(52) U.S. Cl. CPC C07D 515/22; C07B 2200/13

(57) ABSTRACT

The present invention provides pure (1′R,6R,6aR,7R,13S,14S,16R)-5-(acetyloxy)-3′,4′,6,6a,7,13,14,16-octahydro-6′,8,14-trihydroxy-7′,9-dimethoxy-4,10,23-trimethyl spiro[6,16-(epithiopropanoxymethano)-7,13-imino-12H-1,3-dioxolo[7, 8]isoquino[3,2-b][3] benzazocine-20,1′(211)-isoquinolin]-19-one of formula (1) substantially free from one or more impurities selected from intermediate compound of formula (50), cyclic impurity of formula (A), deshydroxy impurity of formula (B) and hydroxy impurity of formula (C).

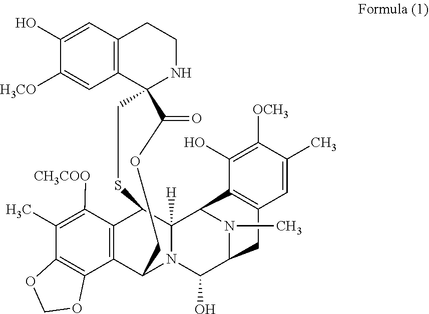

### RELATED APPLICATIONS

This patent application claims the benefit of priority of Indian patent applications bearing numbers IN 201941025538 filed on 27 Jun. 2019 and IN 201941038170 filed on 21 Sep. 2019 which are incorporated herein by reference in its entirety.

### FIELD OF THE INVENTION

(1′R,6R,6aR,7R,13S,14S,16R)-5-(Acetyloxy)-3′,4′,6,6a,7,13,14,16-octahydro-6′,8,14-trihydroxy-7′,9-dimethoxy-4,10,23-trimethylspiro[6,16-(epithiopropanoxymethano)-7,13-imino-12H-1,3-dioxolo[7,8]isoquino[3,2-b][3]benzazocine-20,1′(211)-isoquinolin]-19-one which is commonly known as “Trabectedin” or “Ecteinascidin 743” of formula (1)

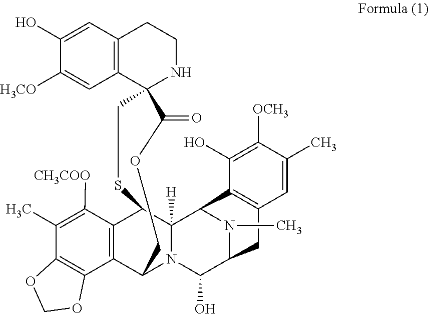

Trabectedin is approved by United States Federal Drug Administration (US FDA) under the brand name of Yondelis® on Oct. 23, 2015 to Janssen products for the treatment of patients with unresectable or metastatic liposarcoma or leiomyosarcoma who received a prior anthracycline-containing regimen. It is available in 1 Mg vial containing powder for IV (Infusion).

The present invention provides an improved process for the preparation and polymorphs thereof.

### BACKGROUND OF THE INVENTION

U.S. Pat. No. 5,089,273 discloses Trabectedin and its process by extraction from marine tunicate Ecteinascidia turbinata.

The drawbacks of the above prior art process is tons of marine tunicates are required to get about one gram of Trabectedin. Further, the prior art process involves complex separation steps, energy-intensive operations, the requirement for large amounts of organic solvents and expensive resins. Hence, the above extraction process becomes tedious, expensive and not viable for commercial scale-up.

U.S. Pat. No. 5,721,362 discloses a process for the preparation of Trabectedin, comprising treating the methoxymethyl ether of formula (49) with trifluoroacetic acid (TFA) in tetrahydrofuran (THF) and water followed by azeotropic distillation with toluene and then purified by flash column chromatography to get the cyano compound of formula (50) which is further treating with silver nitrate (AgNO₃) and water to provide Trabectedin of formula (1).

The main drawback of the above prior art process is growing of cyclic impurity of formula (A) about 4% in compound of formula 50 which is not easy to eliminate with chromatography methods and hence the process required repeated purifications, which results in highly expensive process. Another drawback of the above prior art process is as it only up to milligram level.

The chemical structure of cyclic impurity of formula (A) is as follows:

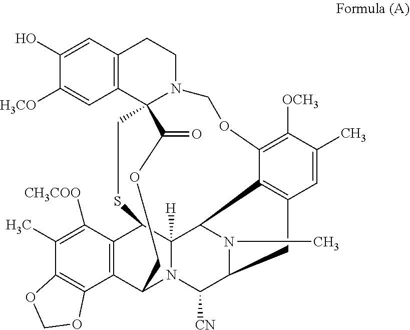

U.S. Pat. No. 8,058,435 discloses purification of Trabectedin using flash chromatography to get pale yellow film.

Acta Crystallographica Section C, 2003, C59, o197-o198 describes 2-propanol disolvate form of Trabectedin.

The main disadvantage of above prior art processes are the use of chromatography methods for the purification of Trabectedin and its intermediate compounds which is cumbersome, tedious, expensive, also requires huge amount of solvents leads to increase in the cost of the production.

U.S. Pat. No. 8,895,557 discloses pharmaceutical composition comprising Trabectedin and a bulking agent such as disaccharide to arrest the development of impurities in long term storage conditions.

Polymorphism occurred because the difference in the physical properties of different solid forms of results from the orientation and intermolecular interactions of adjacent molecules or complexes in the bulk solid. Accordingly, polymorphs are distinct solids sharing the same molecular formula yet having distinct advantageous in physical properties compared to other solid state forms of the same compound.

There is still a need to develop viable, inexpensive, simple and eco-friendly process of Trabectedin. Further, there is a significant need to develop non-solvated and commercial viable polymorphs of Trabectedin of formula (I). Hence, inventors of the present invention had developed an improved process for the preparation of Trabectedin of formula (I) which is simple and circumvent problems in the prior art processes.

### Advantages of the Present Invention

Simple, cost effective and eco-friendly process for the preparation of pure Trabectedin of formula (1) and its intermediate compound of formula (50).
Crystallization of the crude intermediate compound of formula (50) arrests the cyclic impurity of the formula (A) to below about 0.008%.
Pure Trabectedin obtained by the present invention contains less than about 0.01% of intermediate compound of formula (50) and/or substantially free of the cyclic impurity of formula (A), less than about 0.06% of deshydroxy impurity of formula (B).
Pure Trabectedin obtained by the present invention optionally contains less than about 0.05% of hydroxy impurity formula (C).
Pure Trabectedin obtained by the present invention is chemically stable in the absence of bulking agent for about 6 months under long term and accelerated storage conditions.

### BRIEF DESCRIPTION OF THE INVENTION

In one embodiment, the present invention provides Trabectedin of formula (I) substantially free from one or more from intermediate compound of formula (50), cyclic impurity of formula (A), deshydroxy impurity of formula (B) and hydroxy impurity of formula (C).

In second embodiment, the present invention provides a process for the preparation of pure Trabectedin, comprising:
a) providing a solution of crude Trabectedin in a solvent in presence of an acid;
b) neutralizing the obtained solution;
c) isolating pure Trabectedin

In third embodiment, the present invention provides an improved process for the preparation of pure Trabectedin of formula-1, comprising:
a) crystallizing spiro[6,16-(epithiopropanoxymethano)-7,13-imino-12H-1,3-dioxolo[7,8] isoquino[3,2-b][3]benzazocine-20,1′(2′H)-isoquinoline]-14-carbonitrile, 5-(acetyl oxy)-3′,4′,6,6a,7,13,14,16-octahydro-6′,8-dihydroxy-7′,9-dimethoxy-4,10,23-trimethyl-19-oxo-,(1′R,6R,6aR,7R,13S,14R, 16R)- of formula (50) from a solvent,
b) converting the crystalline compound of formula (50) to Trabectedin.

In fourth embodiment, the present invention provides crystalline form of Trabectedin (hereinafter designated as “Form-M”).

In fifth embodiment, the present invention provides a pharmaceutical composition comprising crystalline Form-M of Trabectedin and one or more pharmaceutically acceptable excipients.

### BRIEF DESCRIPTION OF THE DRAWINGS

FIG. 1: Illustrates a characteristic Powdered X-Ray Diffraction (PXRD) pattern of crystalline Form-M of Trabectedin obtained according to example-8.

### DETAILED DESCRIPTION OF THE INVENTION

The term “solvent” used in the present invention refers to “hydrocarbon solvents” selected form n-hexane, n-heptane, cyclohexane, petroleum ether, benzene, toluene, pentane, cycloheptane, methyl cyclohexane, ethylbenzene, m-, o-, or p-xylene, or naphthalene and mixtures thereof; “ether solvents” selected form dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, 1,2-dimethoxy ethane and mixtures thereof; “ester solvents” selected form methyl acetate, ethyl acetate, isopropyl acetate, n-butyl acetate, isobutyl acetate and mixtures thereof; “polar-aprotic solvents selected form dimethylacetamide (DMA), dimethylformamide (DMF), dimethylsulfoxide (DMSO), N-methylpyrrolidone (NMP) and mixtures thereof; “chloro solvents” selected form dichloromethane, dichloroethane, chloroform, carbon tetrachloride and mixtures thereof; “ketone solvents” selected form acetone, methyl ethyl ketone, methyl isobutylketone and mixtures thereof; “nitrile solvents” selected form acetonitrile, propionitrile, isobutyronitrile and mixtures thereof; “alcohol solvents” selected form methanol, ethanol, n-propanol, isopropanol, n-butanol, isobutanol, t-butanol, 2-nitroethanol, 2-fluoroethanol, 2,2,2-trifluoroethanol, ethylene glycol, 1,2-propanediol (propylene glycol), 2-methoxyethanol, 1, 2-ethoxyethanol, diethylene glycol, 1, 2, or 3-pentanol, neo-pentyl alcohol, t-pentyl alcohol, diethylene glycol monoethyl ether, cyclohexanol, benzyl alcohol, phenol, or glycerol and mixtures thereof; “polar solvents” such as water or mixtures thereof.

The term “base” used in the present invention refers to inorganic bases like “alkali metal carbonates” selected from sodium carbonate, potassium carbonate, lithium carbonate and mixtures thereof; “alkali metal bicarbonates” selected from sodium bicarbonate, potassium bicarbonate and mixtures thereof; “alkali metal hydroxides” selected form sodium hydroxide, potassium hydroxide, lithium hydroxide and mixtures thereof; alkali metal hydrides selected form sodium hydride, potassium hydride, lithium hydride and mixtures thereof; alkali metal amides selected from sodium amide, potassium amide, lithium amide or mixtures thereof.

The term “acid” used in the present invention refers to inorganic acids selected from hydrochloric acid (HCl), hydrobromic acid (HBr), hydroiodic acid (HI), sulfuric acid (H₂SO₄); organic acids selected form formic acid, phosphoric acid, orthophosphoric acid, acetic acid, methanesulfonic acid, p-toluenesulfonic acid, trifluoroacetic acid or its aqueous mixtures thereof.

The term “coupling agent” used in the present invention selected from but not limited to 1,1′-carbonyl diimidazole (CDI), N,N′-dicyclohexylcarbodiimide (DCC), N,N′-diisopropyl carbodiimide (DIC), 1-ethyl-3-(3-dimethylaminopropyl)carbodiimide HCl (EDC.HC1), 1-[bis(dimethylamino)methyl ene]-1H-1,2,3-triazolo[4,5-b]pyridinium3-oxid hexafluoro phosphate (HATU), 2-(1H-benzotriazol-1-yl)-1,1,3,3-tetramethyluronium hexafluoro phosphate (HBTU), 1H-benzotriazoliuml-[bis(dimethylamino)methylene]-5chloro-hexafluorophosphate(1-)3-oxide (HCTU), O-(benzotriazol-1-yl)-N,N,N′,N′-tetramethyluronium tetrafluoroborate (TBTU), alkyl/aryl haloformates selected from but not limited to ethyl chloroformate, benzylchloroformate; diphenylphosphoroazidate (DPPA), thionyl chloride, oxalyl chloride, phosphorous oxychloride, phosphorous pentachloride, 4-methyl-2-oxopentanoyl chloride (iBuCOCOCl), (benzotriazol-1-yloxy) tris(dimethylamino) phosphoniumhexafluorophosphate (BOP), benzotriazol-1-yl-oxy tripyrrolidinophosphonium hexafluorophosphate (PyBOP), methane sulfonyl chloride, p-toluenesulfonyl chloride thereof.

The term “protecting group” is selected from but not limited to a group of reagents independently selected such that they are capable of protecting the hydroxy or amine groups of various compounds of the present invention selected from benzyloxycarbonyl (Cbz), 2,2,2-tri chloroethoxy carbonyl(Troc), 2-(trimethyl silyl)ethoxycarbonyl (Teoc), 2-(4-trifluoromethylphenylsulfonyl) ethoxycarbonyl (Tsc), 1-adamantyloxycarbonyl (Adoc), 2-adamantylcarbonyl (2-Adoc), 2,4-dimethylpent-3-yloxycarbonyl (Doc), cyclohexyloxy carbonyl (Hoc), 1,1-dim ethyl-2,2,2-tri chloroethoxy carbonyl (TcBOC), vinyl, 2-chloroethyl, 2-phenyl sulfonyl ethyl, allyl, benzyl, 2-nitrobenzyl, 4-nitrobenzyl, diphenyl-4-pyridylmethyl, N′,N′-dimethylhydrazinyl, methoxymethyl, t-butoxymethyl (Bum), benzyloxymethyl (BOM), 3,4-dihydro-2H-pyranyl(DHP), tetrahydropyranyl (THP), tetrahydrofuranyl (THF), methyl, ethyl, acetyl, benzyl, benzoyl, benzyloxycarbonyl (Cbz), trifluoroacetyl, pivaloyl, allyl, methoxymethyl (MOM), ethoxyethy (EE), methoxyethoxymethyl (MEM), p-methoxybenzyl (PMB), methylthiomethyl (MTM), triphenylmethyl (trityl), methoxytrityl (MMT), dimethoxytrityl (DMT), benzyloxymethyl (BOM), tert.butoxy carbonyl (Boc) thereof; the preferable silyl protecting groups can be selected from but not limited to trialkylsilyl, triarylsilyl, alkyl/aryl silyl such as trimethylsilyl (TMS), triethylsilyl (TES), triisopropylsilyl (TIPS), tert.butyldimethylsilyl (TBS or TBDMS), tri-iso-propylsilyloxymethyl (TOM), tert-butyldiphenylsilyl (TBDPS) thereof.

The term “protecting agent” is selected from but not limited to trialkyl silyl halides such as trimethylsilyl chloride (TMSC1), triethylsilyl chloride (TESC1), triisopropylsilyl chloride (TIPSC1), tert-butyldimethylsilyl chloride (TBDMSC1), tert-butyldiphenylsilyl chloride (TBDPSC1) thereof; trialkyl silyl triflates selected from trimethylsilyl triflate (TMSOTf), triethylsilyl triflate (TESOTf), tert-butyldimethyl silyl triflate (TBDMSOTf or TB SOTO, tert-butyldiphenylsilyl triflate (TBDPSC1) thereof; N,0-bis(trimethylsilyl)acetamide (BSA), hexemethyldisilazane (HMD 5), fluorenylmethyloxy carbonyl chloride (FMOC chloride), dihydropyran, 2-chloro tetrahydrofuran, diazomethane, methyl halides, acetyl chloride, acetic anhydride, benzyl halides, benzoyl chloride, benzoic anhydride, benzyloxycarbonyl chloride, trifluoroacetyl chloride, tert-butyl acetyl chloride, trifluoroacetic anhydride, trifluoroacetic acid, alkyl trifluoroacetates selected form methyl trifluoroacetate, ethyl trifluoroacetate, isopropyl trifluoroacetate, vinyl trifluoroacetate; tert-butyl acetic anhydride, allyl halides, methoxymethyl halides, ethoxyethyl halides, methoxyethoxymethyl halides, p-methoxybenzyl halides, methylthiomethyl halides, trityl halides, benzyloxymethyl halides, di-tert.butyl dicarbonate (DIBOC), alkyl/aryl sulfoni c acids/acid halides/anhydrides selected form methanesulfonyl chloride, ethanesulfonyl chloride, benzenesulfonyl chloride, toluenesulfonyl chloride, methanesulfonic acid, ethanesulfonic acid, p-toluenesulfonic acid, p-toluenesulfonic anhydride thereof.

The term “deprotecting agent” can be selected based on the protecting group employed. The suitable deprotecting agent can be selected from but not limited to acids selected form hydrochloric acid, hydrobromic acid, phosphoric acid, acetic acid, formic acid, trifluoroacetic acid, methane sulfonic acid, p-toluene sulfonic acid, camphor sulfonic acid thereof, bases selected form alkali metal hydroxides, alkali metal carbonates, cesium carbonate/imidazole, alkali metal bicarbonates, ammonia, cerium ammonium nitrate (CAN), 1,8-diazabicyclo[5.4.0]undec-7-ene (DBU), hydrogenating agents selected form Pd, Pd/C, Pd(OH)₂/C (Pearlman's catalyst), palladium acetate, platinum oxide (PtO₂), platinum black, sodium borohydride, BF₃-etherate, Raney-Ni, triethylsilane, trimethylsilyl halides, copper(II) chloride dihydrate and thereof; trifluoromethane sulfonic acid, 2,3-dichloro-5,6-dicyano-1,4-benzoquinone (DDQ), acetyl chloride/methanol, N-iodosuccinimide in methanol; Lewis acids such as AlCl₃, AlBr₃, BBr₃, ZnBr₂, TiCl₄ thereof.

As used herein, the term “substantially free” refers to a compound of the present invention having one or more impurities less than about 0.5% or less than about 0.4% or less than about 0.3% or less than about 0.2% or less than about 0.1% or less than about 0.05% or less than about 0.03 or less than about 0.01%.

As used herein the term “pure” refers to Trabectedin contains purity greater than about 99.50% by high performance liquid chromatography (HPLC) or greater than about 99.70% by HPLC or greater than about 99.80% by HPLC.

In one embodiment, the present invention provides pure Trabectedin of formula (1)

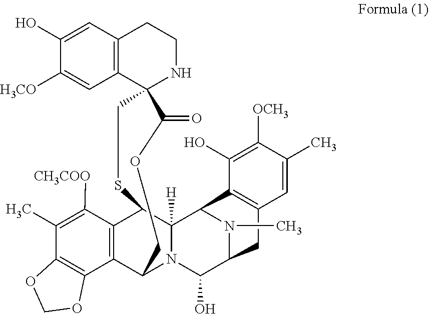

substantially free from one or more impurities selected from intermediate compound of formula (50), cyclic impurity of formula (A), deshydroxy impurity of formula (B) and hydroxy impurity of formula (C).

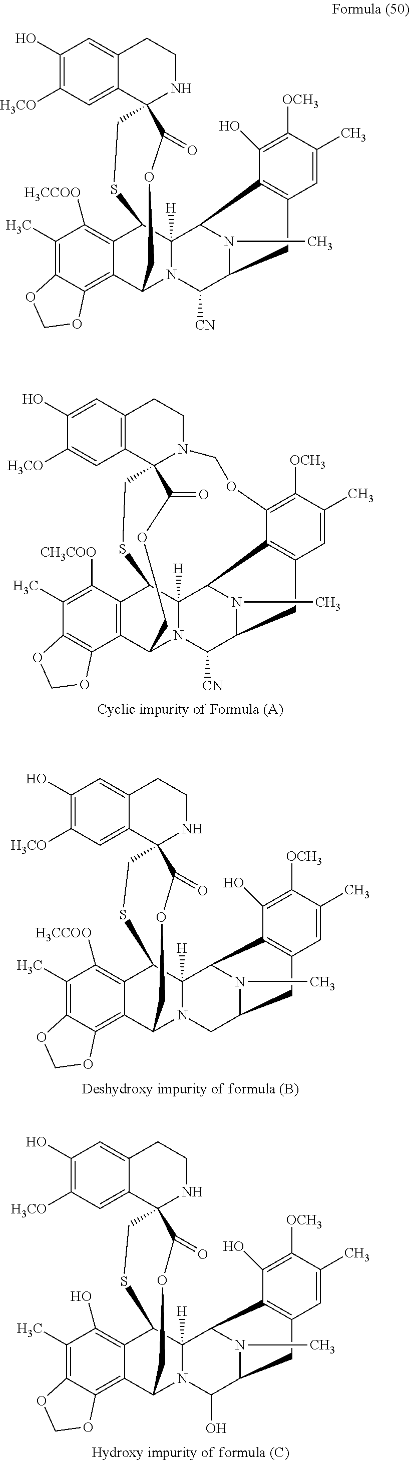

In first aspect of first embodiment, pure Trabectedin contains less than about 0.3% or less than about 0.2% or less than about 0.1% of one or more impurities selected from compound of formula (50), cyclic impurity of formula (A) and deshydroxy impurity of formula (B).

In second aspect of first embodiment, pure Trabectedin obtained by the present invention contains less than about 0.1% or less than about 0.05% or less than about 0.01% of hydroxy impurity of formula (C).

In third aspect of first embodiment, pure Trabectedin obtained by the present invention contains less than about 0.1% or less than about 0.05% or less than about 0.01% of cyclic impurity of formula (A).

In fourth aspect of first embodiment, pure Trabectedin obtained by the present invention is chemically stable for 6 months under long term storage conditions at −15° C. to −25° C. as one or more impurities selected from intermediate compound of formula (50), cyclic impurity of formula (A), deshydroxy impurity of formula (B) and hydroxy impurity of formula (C) do not exceed about 0.3% or about 0.2% or about 0.1%.

In fifth aspect of first embodiment, pure Trabectedin obtained by the present invention is chemically stable for 6 months under accelerated storage conditions at 5±3° C. as one or more impurities selected from intermediate compound of formula (50), cyclic impurity of formula (A), deshydroxy impurity of formula (B) and hydroxy impurity of formula (C) do not exceed about 0.5% or about 0.3% or about 0.1%.

In sixth aspect of first embodiment, pure Trabectedin obtained by the present invention is chemically & physically storage stable in the absence of bulking agent.

In second embodiment, the present invention provides a process for the preparation of pure Trabectedin, comprising:
a) providing a solution of crude Trabectedin in a solvent in presence of an acid;
b) neutralizing the obtained solution;
c) isolating pure Trabectedin
   wherein the acid used in step-a) selected from formic acid, orthophosphoric acid, phosphoric acid, methanesulfonic acid, p-toluenesulfonic acid, nitric acid or its aqueous mixtures thereof; the neutralizing in step-b) is carried out with aqueous dipotassium hydrogen phosphate solution or any other suitable basic medium; “isolating” in step-c) refers to filtration of the precipitated solid and optionally may be dried. Drying may be suitably carried out using equipment such as a tray dryer, vacuum oven, air oven, fluidized bed dryer, spin flash dryer, flash dryer, and thereof, at atmospheric pressure or under reduced pressure. Drying may be carried out at temperatures less than about 50° C., less than about 25° C., less than about 10° C., less than about 5° C., or any other suitable temperatures, in the presence or absence of an inert atmosphere such as nitrogen, argon, neon, or helium. The drying may be carried out for any desired time periods to achieve a desired purity of the product, such as, for example, from about 1 hour to about 15 hours, or longer.

In an aspect of second embodiment, the obtained pure Trabectedin is substantially free from intermediate compound of formula (50), cyclic impurity of formula (A), deshydroxy impurity of formula (B) and hydroxy impurity of formula (C).

In third embodiment, the present invention provides an improved process for the preparation of pure Trabectedin of formula-1, comprising:
c) crystallizing spiro[6,16-(epithiopropanoxymethano)-7,13-imino-12H-1,3-dioxolo[7,8] isoquino[3,2-b][3]benzazocine-20,1′(2′H)-isoquinoline]-14-carbonitrile, 5-(acetyl oxy)-3′,4′,6,6a,7,13,14,16-octahydro-6′,8-dihydroxy-7′,9-dimethoxy-4,10,23-trimethyl-19-oxo-,(1′R,6R,6aR,7R,13S,14R, 16R)- of formula (50) from a solvent,
d) converting the crystalline compound of formula (50) to Trabectedin of formula (1)
   wherein, the solvent used in step-a) for crystallization is selected from nitrile solvents, alcohol solvents, ester solvents, chloro solvents, hydrocarbon solvents or mixtures thereof.

In one aspect of third embodiment, the obtained pure Trabectedin is substantially free from cyclic impurity of formula (A).

In second aspect of third embodiment, the present invention provides an improved process for the preparation of pure Trabectedin of formula-1, comprising:
a) treating the spiro[6,16-(epithiopropanoxymethano)-7,13-imino-12H-1,3-dioxolo[7,8]isoquino[3,2-b][3]benzazocine-20,1′(2′H)-isoquinoline]-14-carbonitrile, 5-(acetyl oxy)-3′,4′,6,6a,7,13,14,16-octahydro-6′-hydroxy-7′,9-dimethoxy-8-(methoxy methoxy)-4,10,23-trimethyl-19-oxo-, (1′R,6R,6 aR,7R,13 S,14R,16R)- of formula (49) with deprotecting agent in non-ether solvent to provide the spiro[6,16-(epithiopropanoxymethano)-7,13-imino-12H-1,3-dioxolo[7,8]isoquino[3,2-b][3] benzazocine-20,1′(2′H)-isoquinoline]-14-carbonitrile,5-(acetyloxy)-3′,4′,6,6a,7,13,14, 16-octahydro-6′,8-dihydroxy-7′,9-dimethoxy-4,10,23-trimethyl-19-oxo-, (1′R,6R,6 aR, 7R,13 S,14R, 16R)- of formula (50),
b) crystallizing spiro[6,16-(epithiopropanoxymethano)-7,13-imino-12H-1,3-dioxolo[7,8] isoquino[3,2-b][3]benzazocine-20,1′(2′H)-isoquinoline]-14-carbonitrile, 5-(acetyl oxy)-3′,4′,6,6a,7,13,14,16-octahydro-6′,8-dihydroxy-7′,9-dimethoxy-4,10,23-trimethyl-19-oxo-,(1′R,6R,6aR,7R,13S,14R, 16R)- of formula (II) from a solvent,
c) converting the compound of formula (50) to Trabectedin of formula (I)
   wherein the deprotecting agent used in step-a) is selected from trifluoroacetic acid, hydrochloric acid, hydrobromic acid, phosphoric acid, acetic acid, formic acid, methane sulfonic acid, p-toluene sulfonic acid; non-ether solvent is selected from nitrile solvents, alcohol solvents, ester solvents, chloro solvents, hydrocarbon solvents and thereof the solvent used in step-b) is selected from nitrile solvents, alcohol solvents, ester solvents, chloro solvents, hydrocarbon solvents thereof.

In third aspect of third embodiment, Trabectedin obtained by the present invention can be further purified by high pressure liquid chromatography (HPLC), preparative HPLC, lyophillization, crystallization, recrystallization or any other methods known in the art.

In fourth aspect of third embodiment, the present invention provides spiro[6,16-(epithiopropanoxymethano)-7,13-imino-12H-1,3-dioxolo[7,8]isoquino[3,2-b][3]benzazocine-20,1′(2′H)-isoquinoline]-14-carbonitrile, 5-(acetyl oxy)-3′,4′,6,6a,7,13,14,16-octahydro-6′,8-dihydroxy-7′,9-dimethoxy-4,10,23-trimethyl-19-oxo-, (1′R,6R,6aR,7R,13 S,14R, 16R)- of formula (50) substantially free of cyclic impurity of formula (A).

In fifth aspect of third embodiment, spiro[6,16-(epithiopropanoxymethano)-7,13-imino-12H-1,3-dioxolo[7,8]isoquino[3,2-b][3]benzazocine-20,1′(2′H)-isoquinoline]-14-carbonitrile, 5-(acetyl oxy)-3′,4′,6,6a,7,13,14,16-octahydro-6′,8-dihydroxy-7′,9-dimethoxy-4,10,23-trimethyl-19-oxo-,(1′R,6R,6aR,7R,13S,14R, 16R)- of formula (50) of the present invention contains less than about 0.5% or less than about 0.3% or less than about 0.1% of an cyclic impurity of formula (A).

In fourth embodiment, the present invention provides crystalline Form-M of Trabectedin.

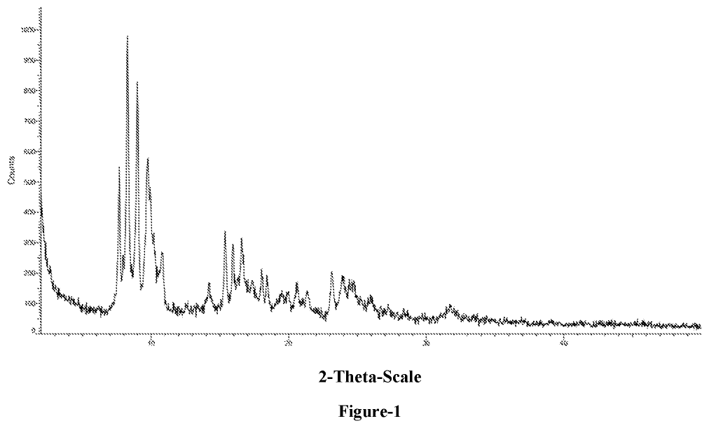

In first aspect of fourth embodiment, the crystalline Form-M of the present invention is characterized by powder X-ray diffraction peaks at about 8.3, 9.0, 15.4, 16.6 and 23.2±0.2 degrees two-theta. Crystalline Form-M of the present invention is further characterized by its X-Ray powder diffraction pattern having additional peaks at about 7.7, 9.7, 14.2, 15.9, 18.0, 18.4 and 20.6±0.2 degrees two-theta. The crystalline Form-M of Trabectedin is further characterized by the Powdered X-Ray Diffraction (PXRD) pattern as illustrated in FIG. 1.

In fifth embodiment, the present invention provides pharmaceutical composition comprising crystalline Form-M of Trabectedin and one or more pharmaceutically acceptable excipients.

In an aspect of the fifth embodiment, the crystalline Form-M of Trabectedin is prepared by the process described in second embodiment.

In another embodiment, the present invention provides pharmaceutical composition comprising therapeutically effective amount of pure Trabectedin according to the present invention & one or more pharmaceutically acceptable excipients wherein, the pharmaceutical excipients is selected from diluents, binders, disintegrants and lubricants. Exemplary diluents include lactose, sucrose, glucose, mannitol, sorbitol, calcium carbonate, microcrystalline cellulose, magnesium stearate, disaccharide and mixtures thereof. Exemplary binders are selected from L-hydroxy propyl cellulose, povidone, HPMC, hydroxylethyl cellulose and pre-gelatinized starch.

Pharmaceutical composition containing pure Trabectedin of the present invention may be prepared by using pharmaceutically acceptable excipients selected from fillers or diluents, bulking agents, binders, wetting agents, disintegrating agents, surface active agents, glidants and lubricants. Various modes of administration of the pharmaceutical composition of the invention can be selected depending on the therapeutic purpose, for example injection preparations.

The injectable pharmaceutical composition may contain one or more additional excipients selected form buffering agents, isotonic agents, antioxidants, preservatives, solubilizes and co-solvents.

Exemplary disintegrants are selected from croscarmellose sodium, cros-povidone, sodium starch glycolate and low substituted hydroxylpropyl cellulose. Exemplary lubricants are selected from sodium stearyl fumarate, magnesium stearate, zinc stearate, calcium stearate, stearic acid, glyceryl behenate and colloidal silicon dioxide.

Starting material “compound of formula (VII) can be prepared according to the methods known from U.S. Pat. No. 5,721,362 or any other methods known in the art.

Powdered X-Ray Diffraction (PXRD) analysis of the crystalline Form-M of pure Trabectedin was carried out using BRUKER-AXS D8 Advance X-Ray diffractometer using Cu-Ka radiation of wavelength 1.5406 A° and at continuous scan speed of 0.03°/min.

High Performance Liquid Chromatography (HPLC) analysis of Trabectedin was carried out by following conditions:

Apparatus: A liquid chromatographic system is equipped with variable wavelength UV detector; Column: Kinetex Biphenyl 100 A°, 250*4.6 mm, 5 μm (or) equivalent; Column temperature: 50° C.; Wavelength: 215 nm; Injection volume: 10.0 μL; Elution: Gradient; Diluent: Methanol; Mobile phase-A: weigh accurately about 2.8 gr of sodium perchlorate monohydrate and transfer into 1000 mL of Milli-Q water followed by pH adjustment with 20% orthophosphoric acid solution. Mobile phase-B: Acetonitrile: Buffer (60:40 v/v)

High Performance Liquid Chromatography (HPLC) analysis of intermediate compound of formula (50) was carried out by following conditions:

Apparatus: A liquid chromatographic system is equipped with variable wavelength UV detector; Column: Kinetex Biphenyl 100 A°, 250*4.6 mm, 5 μm (or) equivalent; Column temperature: 20° C.; Wavelength: 210 nm; Injection volume: 10.0 μL; Elution: Gradient;

Diluent: acetonitrile: methanol; Mobile phase-A: 1.36 gms of potassium dihydrogen phosphate and 0.5 gr of 1-octanesulfonic acid sodium salt in 1000 mL of milli-Q water followed by triethylamine and pH adjustment to 3.0 20% orthophosphoric acid solution Buffer (100%); Mobile phase-B: Acetonitrile: Buffer (70:30 v/v)

The process of the present invention is schematically illustrated in below scheme:

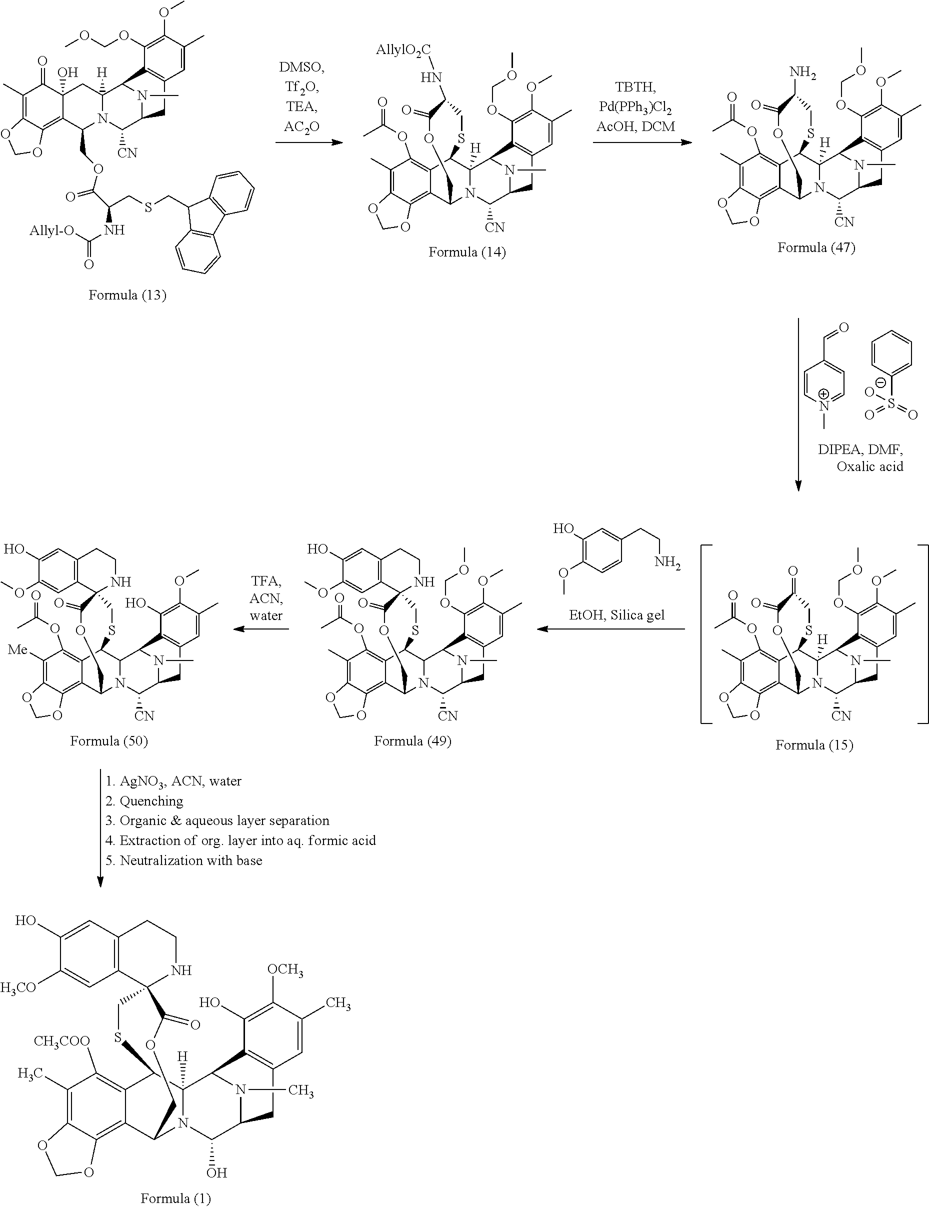

The best mode of carrying out the present invention is illustrated by the below mentioned examples. These examples are for illustrative purposes only and in no way limit the scope of the present invention.

### EXAMPLES

### Example-1: Preparation of Carbamic acid, [5-(acetyloxy)-14-cyano-6,6a,7,13,14,16-hexahydro-9-methoxy-8-(methoxymethoxy)-4,10,23-trimethyl-19-oxo-6,16-(epithioprop anoxymethano)-7,13-imino-12H-1,3-dioxolo[7,8] isoquino[3,2-b][3] benzazocin-20-yl]-,2-propenyl ester,[6R-(6R*,6aR*,7R*,13S*,14R*,16R*,20R*)]-9(CI) of formula (VI)

Dichloromethane (250 ml) was added to trifluoromethane sulfonicanhydride (4.2 gms) and molecular sieves (50 gms). A solution of dimethylsulfoxide in dichloromethane was added to the above mixture at −75±5° C. A solution of (2R)-((6aS,7R,13S,14R,16R)-14-Cyano-5a-hydroxy-9-methoxy-8-(methoxymethoxy)-4,10,17-trimethyl-5-oxo-5a,6,6a,7,12,13,14,16-octahydro-5H-7,13-epiminobenzo[4,5] azocino[1,2-b][1,3] dioxolo[4,5-h]isoquinolin-16-yl)methyl3-(((9H-fluoren-9-yl)methyl)thio)-2-(((allyloxy)carbonyl)amino)propanoate of formula (VII) (10 gms) and molecular sieves in dichloromethane was added to the above obtained reaction mixture and stirred for 30 min at −40±5° C. Triethylamine (10.3 ml), tert-butanol (9.4 ml), 2-tert-butyl-1,1,3,3-tetramethyl guanidine (37.0 ml), acetic anhydride (15.5 ml) and dichloromethane were added to the above obtained reaction mixture at −20±5° C. and stirred for 20 min. Quenched the reaction mixture with water. Separated the layers and distilled off the solvent from the organic layer followed by column purification (cyclohexane and ethyl acetate) of the obtained compound to get the title compound

Yield: 4.84 gms

### Example-2: Preparation of (6R,6aR,7R,13S,14R,16R,20R)-5-(acetyloxy)-20-amino-6,6a,7,13,14,16-hexahydro-9-methoxy-8-(methoxymethoxy)-4,10,23-trimethyl-19-oxo-6,16-(epithiopropanoxymethano)-7,13-imino-12H-1,3-dioxolo[7,8]isoquino[3,2-b][3] benzazocine-14-carbonitrile of formula (47)

Carbamic acid, [5-(acetyloxy)-14-cyano-6,6a,7,13,14,16-hexahydro-9-methoxy-8-(methoxymethoxy)-4,10,23-trimethyl-19-oxo-6,16-(epithiopropanoxymethano)-7,13-imino-12H-1,3-dioxolo[7,8]isoquino[3,2-b][3]benzazocin-20-yl]-,2-propenyl ester, [6R-(6R*,6aR*, 7R*,13S*,14R*,16R*,20R*)]-9(CI) (4 gms) was dissolved in dichloromethane (800 ml). Bis triphenylphosphine palladium (II) chloride (0.15 gms), acetic acid (3.0 ml), tributyltinhydride (3.20 ml) were added to the above obtained mixture and stirred for 1 hr under argon atmosphere. Quenched the reaction mixture with thiophenol resin and filtered through hy-flow bed. Aqueous potassium fluoride solution was added to the obtained filtrate and stirred for 10 min. Separated the layers and washed the organic layer with water. Separated the layers and distilled off the solvent from the organic layer followed by column purification (cyclohexane and ethyl acetate) of the obtained compound to get the title compound Yield: 3 gms.

### Example-3: Preparation of spiro[6,16-(epithiopropanoxymethano)-7,13-imino-1211-1,3-dioxolo[7,8] isoquino[3,2-b][3]benzazocine-20,1′(2′H)-isoquinoline]-14-carbonitrile, 5-(acetyloxy)-3′,4′,6,6a,7,13,14,16-octahydro-6′-hydroxy-7′,9-dimethoxy-8-(methoxy methoxy)-4,10,23-trimethyl-19-oxo-,(1′R,6R,6aR,7R,13S,14R,16R)- of formula (49)

(6R,6aR,7R,13S,14R,16R,20R)-5-(Acetyloxy)-20-amino-6,6a,7,13,14,16-hexahydro-9-methoxy-8-(methoxymethoxy)-4,10,23-trimethyl-19-oxo-6,16-(epithiopropanoxy methano)-7,13-imino-12H-1,3-dioxolo[7,8]isoquino[3,2-b][3]benzazocine-14-carbonitrile (5 gms) was added to dimethylformamide (250 ml). 4-Formyl-1-methylpyridin-1-ium benzenesulfonate (30.0 gms) was added to the above mixture at 25-30° C. and stirred for 45 min at same temperature. Basified the above reaction mixture with diisopropylethylamine (19.4 ml) at 25-30° C. and stirred for 2 hrs. Cooled the reaction mixture to 0-5° C. and acidified with aqueous oxalic acid solution. Raised the temperature to 25-30° C. and stirred for 2 hrs. Quenched the reaction mixture with aqueous sodium bicarbonate solution and dilute with methyl tert-butyl ether. Separated the organic and aqueous layers and extracted the aqueous layer with methyl tert-butyl ether. Combined the total organic layers and washed with water. Separated the organic layer and distilled off the solvent completely from the organic layer under reduced pressure. Dissolved the obtained compound in ethanol (35.0 ml) at 25-30° C. and added 5-(2-aminoethyl)-2-methoxyphenol (1.14 gms) and silica gel (5.0 gms) to it followed by stirred for 2 hrs. Filtered the reaction mixture and washed with dichloromethane. Washed the filtered mls with water and settle for separation of the layers. Distilled off the solvent from organic layer under the reduced pressure. Dissolved the obtained residue in ethanol and added water. Filtered the obtained compound and washed with water. N-Heptane was added to the obtained material and stirred for 45 min. Filtered the solid, washed with n-heptane and then dried to afford the title compound.

Yield: 2.66 gms, MR: 214.1-215.2° C.

### Example-4: Preparation of spiro[6,16-(epithiopropanoxymethano)-7,13-imino-12H-1,3-dioxolo[7,8] isoquino[3,2-b][3]benzazocine-20,r(2′H)-isoquinoline]-14-carbonitrile, 5-(acetyloxy)-3′,4′,6,6a,7,13,14,16-octahydro-6′,8-dihydroxy-7′,9-dimethoxy-4,10,23-trimethyl-19-oxo-, (1′R,6R,6aR,7R,13S,14R, 16R)- of formula (50)

Spiro[6,16-(epithiopropanoxymethano)-7,13-imino-12H-1,3-dioxolo[7,8] isoquino[3,2-b][3]benzazocine-20,1′(2′H)-isoquinoline]-14-carbonitrile, 5-(acetyloxy)-3′,4′,6,6a,7,13, 14,16-octahydro-6′-hydroxy-7′,9-dimethoxy-8-(methoxymethoxy)-4,10,23-trimethyl-19-oxo-,(1′R,6R,6aR,7R,13S,14R,16R)-(9CI) of formula (49) (2.0 gms) was added to acetonitrile (22.4 ml) and water (11.0 ml) at 25-30° C. Trifluoro acetic acid (66.6 ml) was added to the above mixture at 0-5° C. and raised the temperature of the mixture to 25-30° C. and then stirred for 5 hrs at same temperature. Quenched the reaction mixture with aqueous sodium bicarbonate solution. Washed the obtained mixture with dichloromethane and separated the organic and aqueous layers. Extracted the aqueous layer with mixture of ethanol and dichloromethane. Combined the total organic layers and washed with water. Charcoal (2.0 gms) was added to the organic layer and stirred for 30 min at 25-30° C. Filtered the obtained mixture through by-flow bed, washed with dichloromethane and ethanol and distilled off the solvent from the filtered mls. Dissolved the obtained crude compound in ethanol (20.0 ml) at 25-30° C. and stirred for 30 min. Filtered the precipitated solid and washed with ethanol. n-Heptane (20.0 ml) was added to above obtained compound and stirred for 45 min at 25-30° C. Filtered the solid and washed with n-heptane and then dried to afford the title compound.

Yield: 2 gms, cyclic impurity of formula (A): 3.61%, purity: 84.77% by HPLC.

### Example-5: Preparation of pure spiro[6,16-(epithiopropanoxymethano)-7,13-imino-1211-1,3-dioxolo[7,8] isoquino[3,2-b][3] benzazocine-20,1′ (2′H)-isoquinoline]-14-carbonitrile, 5-(acetyloxy)-3′,4′,6,6a,7,13,14,16-octahydro-6′,8-dihydroxy-7′,9-dimethoxy-4,10,23-trimethyl-19-oxo-, (1′R,6R,6aR,7R,13S,14R, 16R)- of formula (50)

Dissolved crude spiro[6,16-(epithiopropanoxymethano)-7,13-imino-12H-1,3-dioxolo[7,8]isoquino[3,2-b][3]benzazocine-20,1′(2′H)-isoquinoline]-14-carbonitrile, 5-(acetyl oxy)-3′,4′,6,6a,7,13,14,16-octahydro-6′,8-dihydroxy-7′,9-dimethoxy-4,10,23-trimethyl-19-oxo-, (1′R,6R,6aR,7R,13S,14R, 16R)- (2 gms) in ethanol (20.0 ml) at 25-30° C. and stirred for 30 min. Filtered the precipitated solid and washed with ethanol. n-Heptane (20.0 ml) was added to above obtained compound and stirred for 45 min. Filtered the solid and washed with n-heptane and then dried to afford the title compound.

Yield: 1.33 gms, MR: 223.6° C.-224.8° C., purity: 97.61% by HPLC, cyclic impurity of formula (A): 0.23% by HPLC

### Example-6: Preparation of pure spiro[6,16-(epithiopropanoxymethano)-7,13-imino-12H-1,3-dioxolo[7,8] isoquino[3,2-b][3] benzazocine-20,1′ (2′H)-isoquinoline]-14-carbonitrile, 5-(acetyloxy)-3′,4′,6,6a,7,13,14,16-octahydro-6′,8-dihydroxy-7′,9-dimethoxy-4,10,23-trimethyl-19-oxo-, (1′R,6R,6aR,7R,13S,14R, 16R)- of formula (50)

Dissolved crude spiro[6,16-(epithiopropanoxymethano)-7,13-imino-12H-1,3-dioxolo[7,8]isoquino[3,2-b][3]benzazocine-20,1′(2′H)-isoquinoline]-14-carbonitrile, 5-(acetyl oxy)-3′,4′,6,6a,7,13,14,16-octahydro-6′,8-dihydroxy-7′,9-dimethoxy-4,10,23-trimethyl-19-oxo-, (1′R,6R,6aR,7R,13S,14R, 16R)- (1.5 gms) in isopropanol (10.0 ml) at 25-30° C. and stirred for 45 min. Filtered the precipitated solid, washed with isopropanol and then dried to afford the title compound.

Yield: 0.98 gms, MR: 223.6° C.-224.8° C., purity: 97.4% by HPLC, cyclic impurity of formula (A): 0.06% by HPLC

### Example-7: Preparation of pure spiro[6,16-(epithiopropanoxymethano)-7,13-imino-12H-1,3-dioxolo[7,8] isoquino[3,2-b][3] benzazocine-20,1′ (2′H)-isoquinoline]-14-carbonitrile, 5-(acetyloxy)-3′,4′,6,6a,7,13,14,16-octahydro-6′,8-dihydroxy-7′,9-dimethoxy-4,10,23-trimethyl-19-oxo-, (1′R,6R,6aR,7R,13S,14R, 16R)- of formula (50)

Spiro[6,16-(epithiopropanoxymethano)-7,13-imino-12H-1,3-dioxolo[7,8] isoquino[3,2-b][3]benzazocine-20,1′(2′H)-isoquinoline]-14-carbonitrile, 5-(acetyloxy)-3′,4′,6,6a,7,13, 14,16-octahydro-6′-hydroxy-7′,9-dimethoxy-8-(methoxymethoxy)-4,10,23-trimethyl-19-oxo-,(1′R,6R,6aR,7R,13S,14R,16R)-(9CI) of formula (III) (7.0 gms) was added to acetonitrile (78.4 ml) and water (38.5 ml) at 25-30° C. Trifluoro acetic acid (0.23 Lt) was added to the above mixture at 0-5° C. and raised the temperature of the mixture to 25-30° C. and then stirred for 5 hrs. Diluted the reaction mixture with water. Quenched the reaction mixture with aqueous sodium bicarbonate solution. Diluted the above obtained mixture with dichloromethane and separated the organic and aqueous layers. Extracted the aqueous layer with mixture of ethanol and dichloromethane. Combined the total organic layers and washed with water. Charcoal (7.0 gms) was added to the organic layer and stirred for 30 min at 25-30° C. Filtered the obtained mixture through by-flow bed, washed with dichloromethane, ethanol and distilled off the solvent from the filtered mls. Dissolved the obtained crude compound in ethanol (70.0 ml) at 25-30° C. and stirred for 30 min. Filtered the precipitated solid and washed with ethanol. n-Heptane (70.0 ml) was added to above obtained compound and stirred for 45 min at 25-30° C. Filtered the solid and washed with n-heptane and then dried to afford the title compound.

Yield: 5.3 gms, cyclic impurity of formula (A): Not detected; purity: 99.73% by HPLC.

### Example-8: Preparation of (1′R,6R,6aR,7R,13S,14S,16R)-5-(acetyloxy)-3′,4′,6,6a,7,13,14,16-octahydro-6′,8,14-trihydroxy-7′,9-dimethoxy-4,10,23-trimethylspiro[6,16-(epithiopropanoxymethano)-7,13-imino-12H-1,3-dioxolo[7,8] isoquino[3,2-b][3] benzazocine-20,1′(2′H)-isoquinolin]-19-one (Trabectedin)

Spiro[6,16-(epithiopropanoxymethano)-7,13-imino-12H-1,3-dioxolo[7,8]isoquino[3,2-b][3]benzazocine-20,1′(2′H)-isoquinoline]-14-carbonitrile, 5-(acetyloxy)-3′,4′,6,6a,7,13, 14,16-octahydro-6′,8-dihydroxy-7′,9-dimethoxy-4,10,23-trimethyl-19-oxo-, (1′R,6R,6aR,7R, 13S,14R, 16R)- (1 gm) was dissolved in acetonitrile (90 ml) and water (60 ml). Silver nitrate (5.47 gms) was added to the above mixture and stirred for 12 hrs at 25-30° C. Quenched the reaction mixture with aqueous sodium chloride solution and aqueous sodium bicarbonate solution. Separated the organic, aqueous layers and extracted the aqueous layer with methyl tert-butyl ether. Combined the total organic layer and washed with aqueous sodium chloride solution. Separated the organic layer and washed with water. Separated the organic layer and dried over sodium sulfate. Distilled off the solvent from the organic layer under reduced pressure. The obtained compound was dissolved in pre-cooled dichloromethane (1000 ml) and acidified with aqueous orthophosphoric acid solution. Separated the aqueous and organic layers and washed the aqueous layer with dichloromethane. Separated the aqueous layer and neutralized it with aqueous dipotassium hydrogen phosphate solution. Separated the organic and aqueous layer and extracted the aqueous layer with methyl tert-butyl ether. Combined the total organic layers and distilled off the solvent under reduced pressure and then dried to afford the title compound. (Yield: 700 mg)

### Example-9: Preparation of Pure Trabectedin of Formula (I)

Spiro[6,16-(epithiopropanoxymethano)-7,13-imino-12H-1,3-dioxolo[7,8] isoquino[3,2-b][3]benzazocine-20,1′(2′H)-soquinoline]-14-carbonitrile, 5-(acetyloxy)-3′,4′,6,6a,7,13, 14,16-octahydro-6′,8-dihydroxy-7′,9-dimethoxy-4,10,23-trimethyl-19-oxo-, (1′R,6R,6aR,7R,13S,14R,16R)- (2.5 gms) is dissolved in acetonitrile (340 ml) at 20-25° C. Water (110 ml) and silver nitrate (16.5 gms) were added to the above mixture at 20-25° C. and stirred for 15-16 hrs. Quenched the reaction mixture with mixture of aqueous sodium bicarbonate and aqueous sodium chloride solution at 10-15° C. Extracted the reaction mixture with dichloromethane. Separated the layers and extracted the aqueous layer with dichloromethane. Organic layer is washed with mixture of aqueous sodium bicarbonate and aqueous sodium chloride solution followed by washed with water. Extracted the compound from organic layer with aqueous formic acid solution. Separated the layers and washed the aqueous layer with methyl tert butyl ether. Separated the layers and neutralized the aqueous layer with aqueous di potassium hydrogen phosphate solution. Filtered the precipitated solid, washed with water and then dried to afford the title compound.

(Yield: 1.24 gms, purity: 99.78% by HPLC, deshydroxy impurity formula (B): 0.08%, intermediate impurity of formula (50): 0.01%).

The PXRD pattern of the obtained pure Trabectedin (crystalline Form-M) is illustrated in FIG. 1.

### Example-10: Preparation of Pure Trabectedin of Formula (I)

Spiro[6,16-(epithiopropanoxymethano)-7,13-imino-12H-1,3-dioxolo[7,8] isoquino[3,2-b][3]benzazocine-20,1′(2′H)-isoquinoline]-14-carbonitrile, 5-(acetyloxy)-3′,4′,6,6a,7,13, 14,16-octahydro-6′,8-dihydroxy-7′,9-dimethoxy-4,10,23-trimethyl-19-oxo-, (1′R,6R,6aR,7R,13S,14R,16R)- (5 gms) dissolved in acetonitrile (0.68 Lt) at 20-25° C. Water (0.22 Lt) and silver nitrate (33 gms) were added to the above mixture at 20-25° C. and stirred for 16 hrs. Quenched the reaction mixture with mixture of aqueous sodium bicarbonate and aqueous sodium chloride solution at 10-15° C. Separated the organic, aqueous layers and extracted the aqueous layer with dichloromethane. Combined the total organic layers and washed with mixture of aqueous sodium bicarbonate and aqueous sodium chloride solution followed by water. Separated the organic and aqueous layers. Extracted the organic layer with aqueous formic acid solution. Separated the aqueous and organic layers and washed the aqueous layer with methyl tert butyl ether. Separated the aqueous and organic layers and neutralized the aqueous layer with aqueous di potassium hydrogen phosphate solution. Filtered the precipitated solid, washed with water and then dried to afford the title compound.

Yield: 2.2 gms, purity: 99.80% by HPLC, deshydroxy impurity of formula (B): 0.06%, intermediate impurity of formula (50): 0.01%, cyclic impurity of formula (A): Not detected.

1. Pure Trabectedin of formula (1) 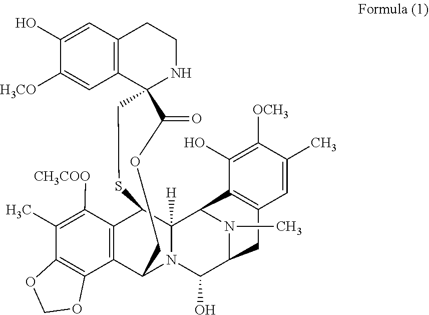
which is substantially free from one or more impurities selected from intermediate compound of formula (50), cyclic impurity of formula (A), deshydroxy impurity of formula (B) and hydroxy impurity of formula (C).

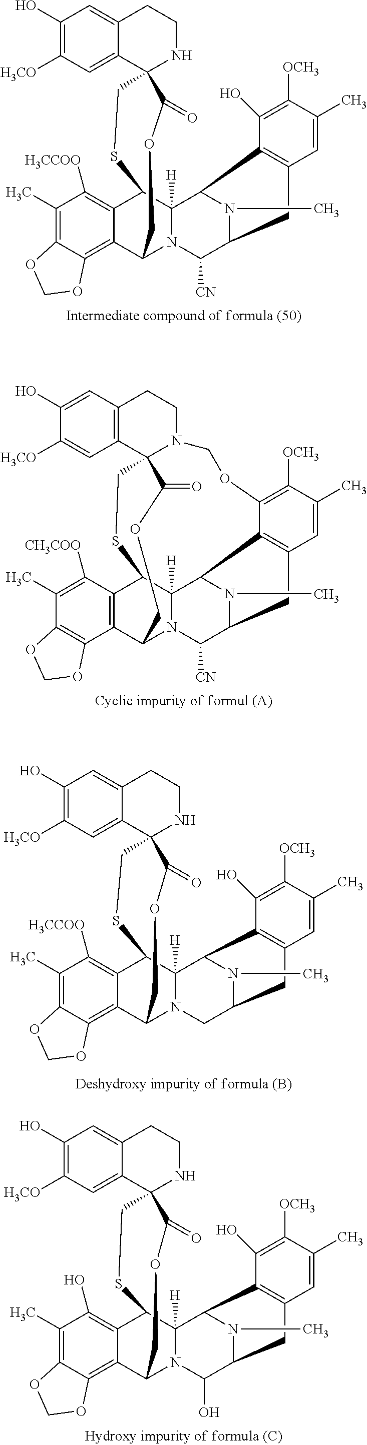

2. Pure Trabectedin of claim 1, contains less than about 0.3% or less than about 0.2% or less than about 0.1% of one or more impurities selected from intermediate compound of formula (50), cyclic impurity of formula (A), deshydroxy impurity of formula (B) and hydroxy impurity of formula (C).

3. Pure Trabectedin of claim 1, having less than about 0.1% or less than about 0.05% or less than about 0.01% of hydroxy impurity of formula (C).

4. Pure Trabectedin of claim 1, having less than about 0.1% or less than about 0.05% or less than about 0.01% of cyclic impurity of formula (A).

5. Pure Trabectedin of claim 1, prepared by a process comprising:
a) providing a solution of crude Trabectedin in a solvent in presence of an acid;
b) neutralizing the obtained solution;
c) isolating pure Trabectedin.

6. The process of claim 5, wherein the acid used in step-a) is selected from formic acid, orthophosphoric acid, phosphoric acid, methanesulfonic acid, p-toluenesulfonic acid, nitric acid or its aqueous mixtures thereof.

7. The process of claim 5, wherein the neutralization in step-b) is carried out with a base.

8. The process of claim 7, wherein the base is aqueous dipotassium hydrogen phosphate solution.

9. The process of claim 5, wherein the isolation in step-c) is carried out by filtration.

10. An improved process for the preparation of pure Trabectedin of formula-1, 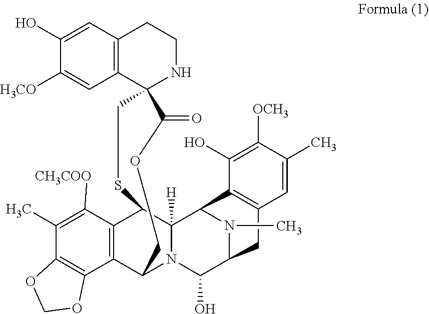
comprising:
a) crystallizing spiro[6,16-(epithiopropanoxymethano)-7,13-imino-12H-1,3-dioxolo[7,8] isoquino[3,2-b][3]benzazocine-20,1′(2′H)-isoquinoline]-14-carbonitrile, 5-(acetyl oxy)-3′,4′,6,6a,7,13,14,16-octahydro-6′,8-dihydroxy-7′,9-dimethoxy-4,10,23-trimethyl-19-oxo-,(1′R,6R,6aR,7R,13S,14R, 16R)- of formula (50) from a solvent, b) converting the crystalline compound of formula (50) to pure Trabectedin.

11. The process of claim 10, wherein the solvent used in step-a) is selected from nitrile solvents, alcohol solvents, ester solvents, chloro solvents, hydrocarbon solvents or mixtures thereof.

12. The process of claim 10, wherein pure Trabectedin is substantially free from cyclic impurity of formula (A).

13. The process of claim 10, wherein the compound of formula (50) is prepared by a process comprising, treating the spiro[6,16-(epithiopropanoxymethano)-7,13-imino-12H-1,3-dioxolo[7,8]isoquino[3,2-b][3]benzazocine-20,1′(2′H)-isoquinoline]-14-carbonitrile, 5-(acetyl oxy)-3′,4′,6,6a,7,13,14,16-octahydro-6′-hydroxy-7′,9-dimethoxy-8-(methoxy methoxy)-4,10,23-trimethyl-19-oxo-, (1′R,6R,6 aR,7R,13 S,14R,16R)- of formula (49) with a deprotecting agent in non-ether solvent to provide the spiro[6,16-(epithiopropanoxymethano)-7,13-imino-12H-1,3-dioxolo[7,8]isoquino[3,2-b][3] benzazocine-20,1′(2′H)-isoquinoline]-14-carbonitrile,5-(acetyloxy)-3′,4′,6,6a,7,13,14,16-octahydro-6′,8-dihydroxy-7′,9-dimethoxy-4,10,23-trimethyl-19-oxo-,(1′R,6R,6aR, 7R,13S,14R, 16R)- of formula (50).

14. The process of claim 13, wherein the deprotecting agent used in step-a) is selected from trifluoroacetic acid, hydrochloric acid, hydrobromic acid, phosphoric acid, acetic acid, formic acid, methane sulfonic acid, p-toluene sulfonic acid; non-ether solvent is selected from nitrile solvents, alcohol solvents, ester solvents, chloro solvents, hydrocarbon solvents and thereof.

15. Spiro[6,16-(epithiopropanoxymethano)-7,13-imino-12H-1,3-dioxolo[7,8]isoquino[3,2-b][3]benzazocine-20,1′(2′H)-isoquinoline]-14-carbonitrile, 5-(acetyloxy)-3′,4′,6,6a,7,13,14,16-octahydro-6′,8-dihydroxy-7′,9-dimethoxy-4,10,23-trimethyl-19-oxo-,(1′R,6R,6aR,7R,13S,14R, 16R)- of formula (50) substantially free from cyclic impurity of formula (A).

16. Spiro[6,16-(epithiopropanoxymethano)-7,13-imino-12H-1,3-dioxolo[7,8]isoquino[3,2-b][3]benzazocine-20,1′(2′H)-isoquinoline]-14-carbonitrile, 5-(acetyloxy)-3′,4′,6,6a,7,13,14,16-octahydro-6′,8-dihydroxy-7′,9-dimethoxy-4,10,23-trimethyl-19-oxo-, (1′R,6R,6aR,7R,13 S,14R, 16R)- of claim 15, contains less than about 0.5% or less than about 0.3% or less than about 0.1% of cyclic impurity of formula (A).

17. Crystalline Form-M of Trabectedin of formula (1).

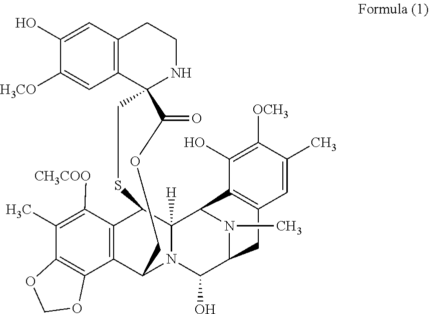

18. The crystalline Form-M of claim 17, characterized by powder X-ray diffraction peaks at about 8.3, 9.0, 15.4, 16.6 and 23.2±0.2 degrees two-theta.

19. The crystalline Form-M of claim 17 further characterized by its X-Ray powder diffraction pattern having additional peaks at about 7.7, 9.7, 14.2, 15.9, 18.0, 18.4 and 20.6±0.2 degrees two-theta.

20. The crystalline Form-M of Trabectedin of claim 17 is further characterized by the Powdered X-Ray Diffraction (PXRD) pattern as illustrated in FIG. 1.

21. Pharmaceutical composition comprising Trabectedin Form-M and one or more pharmaceutically acceptable excipients.

22. Pharmaceutical composition comprising pure Trabectedin and one or more pharmaceutically acceptable excipients
